(19) United States
(12) Patent Application Publication
(10) Pub. No.: US 2015/0182373 A1
(43) Pub. Date: Jul. 2, 2015

(54) Oral Exercise Appliance

(71) Applicant: Todd D. Morgan, DMD, Encinitas, CA (US)

(72) Inventor: Todd D. Morgan, DMD, Encinitas, CA (US)

(21) Appl. No.: 14/146,630

(22) Filed: Jan. 2, 2014

(51) Int. Cl. A61F 5/56

(52) U.S. Cl. CPC A61F 5/566

(57) ABSTRACT

A dental device relating to a mouth-engageable component positioned on the palatal portion of the mouth, and configured for employment by the user for exercising and strengthening the pharyngeal muscles. Repetitive use results in a strengthening of the muscles relating to tongue movement and provides an increase of the area of their pharyngeal airway. Such an increase can provide a reduction in the severity of snoring and sleep apnea.

### BACKGROUND OF THE INVENTION

1. Field of the Invention

The present invention relates to dental devices. More particularly, the disclosed device relates to a mouth-engageable component configured for employment by the user for exercising and strengthening the pharyngeal muscles. Repetitive use by the user results in a strengthening and increase in endurance of the muscles relating to tongue movement and a toning of the muscles involved with such, thereby providing a concurrent increase of the area of their pharyngeal airway. Such an increase can provide a reduction in the severity of snoring and sleep apnea.

2. Prior Art

Sleep apnea occurs when a person momentarily stops breathing or suffers diminished breathing during sleep. Research conventionally has shown such is primarily caused by impaired air passages for breathing while sleeping. The severity of detrimental daytime symptoms caused by sleep apnea varies with the frequency of occurrence, and the severity of snoring and cessation of breathing during such occurrences.

In many patients, the cessation or pause in their breathing, and the resulting lack of oxygen communicated to their body through respiration, may last from a few seconds or in severe cases, the better part of a minute. In many patients, cessation of breathing, lessening of oxygen to the body can occur up to 30 times or more an hour. During such episodes, the patient ceases breathing and the normally reflex action of breathing only begins again when the patient partially awakens due to oxygen deprivation which causes a gasping for air.

In addition to the lower levels of blood oxygen available to the patient's body and brain, awaking from normal sleep many times an hour, during periods when the patient should be sleeping, has a dramatic effect on their daytime awareness. Further, this sleep depravation impairs their concentration, and the ability to stay awake. Indeed it is well known that many drivers who fall asleep at the wheel, suffer from sleep apnea and an ongoing lack of evening sleep.

One widely suffered type of sleep apnea, known as obstructive sleep apnea (OSA), results from blocked airflow passages in the throat during sleep. Such obstructive blockages result from a narrowing in the pharyngeal airway. Such narrowing occurs during natural muscle relaxation of the patient during sleep.

In physically fit persons, such relaxation is generally tolerated well. However, anatomical factors such as obesity, weak pharyngeal muscles, age, and repeated lack of sleep on previous evenings, can also contribute to a worsening of OSA. In overweight individuals, the narrowing of breathing passages is primarily due to a lack of muscle tone in the muscles which support and help maintain the airway from the mouth and nose to the lungs. Indeed, in many patients, a total closure of this vital air passageway to the lungs results from a lack of toned muscles in the throat region with which to maintain their breathing airway open during sleep.

Closure or narrowing of this air passageway severely reduces or actually prevents air passage to the lungs during breathing while asleep. As noted, a reopening of this air passage frequently only occurs when the patient is jolted to a semi conscious state by the severely low oxygen levels in their bloodstream. The low oxygen levels cause a reflex reaction to awaken sufficiently to intentionally cause their throat muscles to realign and allow air passage. This reflex causes a resulting gasp for air much like a swimmer who has been underwater too long. As noted, this sleep and waking process can repeat itself hundreds of times in an eight-hour stretch of attempted sleep.

In patients suffering from an obstructive blockage due to muscle relaxation, the narrowing of the air passages also frequently results in a loud snoring noise. Indeed, in some patients, their own snoring can rise to decibel levels of noise where the sound actually arouses the patient from needed sleep. Thus, for a majority of patients, ongoing severe sleep apnea presents a serious risk of injury arising from interrupted oxygen supply to the body, lack of REM sleep, and severe problems with anyone unfortunate enough to be sharing sleeping accommodations with them.

Conventional treatments for patients suffering from sleep apnea involve significant lifestyle changes. These can include avoiding alcohol or muscle relaxants in order to help lessen the atrophy of throat muscles during sleep. Further, many patients are instructed to lose weight in order to try and reduce the fat content of their body and fatty tissue surrounding the muscles of the throat, in order to widen their air passages. Other suggested treatment regimens include smoking cessation which for many patients is virtually impossible.

Treatments other than patient lifestyle and diet changes are also available. One such medical treatment is provided through the employment of air pumps and masks required for positive airway pressure (PAP) therapy devices. However, the masks required for communication of positive air pressure through the nose and mouth, are extremely uncomfortable, can cause rashes, and in some patients cause claustrophobic symptoms and resulting anxiety. Still further, the widely used PAP systems are primarily employable at home because the pumps and hoses and masks required for function do not travel well.

An additional problem with PAP systems is one of bedtime company from the spouse or a significant other of the sleep apnea sufferer. The noise from the engaged pumping devices and the continual rolling and movement of the masked patient, generally cause the patient's partner to remove him or herself to another quieter room. Such isolation of the sleep apnea sufferer and their partner can result in serious marital and personal problems over time.

Another known mode for treating sleep apnea is the employment of a mouth-engaged appliance to reposition the jaw. Using such devices the patient's jaw is mechanically maintained in a forward but unnatural positioning relative to the upper jaw. This intentional mis-alignment of their bite, pulls the tongue forward or toward their mouth to maintain a forward (thrust) positioning thereby physically widening the patient's airway rearward of their tongue by moving that forward. Such a physical widening reduces the obstruction of the pharyngeal airway when those muscles assume their relaxed state and thus reduces the occurrence of the blockage of airflow during sleep.

However, for many individuals, the many hours of unnatural jaw positioning of their natural bite positioning can result in jaw pain and stiffness. Further, for some patients, even wearing the jaw-positioning appliance, naturally maintaining a forward positioning of their tongue is unachievable due to prior habits, weak pharyngeal muscles, or other medical conditions.

As a result of the shortcomings in mouth-worn sleep apnea treatments the prior art has advanced and taught many methods and devices for tongue positioning and tongue thrust correction via patient worn devices, muscle training devices, isometric exercises, and other components to strengthen the related muscles.

US Pat. No. 5,052,409 to Tepper is one of many examples of art which teaches a device for tongue thrust positioning. The device taught by Tepper includes an orally engaged device employing a biasing of a thin arched member against the dorsum of the tongue at or near the base of the tongue. The provided urging of the tongue encourages a proper static positioning of the anterior part of the tongue during sleep, to contact the rugae for a proper proprioceptive feel and to provide an airway for proper nasal breathing.

This is accomplished by stimulating the dorsum of the tongue to compress against the soft palate. However, one skilled in the art can easily discern that the configuration of the device taught by Tepper, which in an as used mouth position contacts the dorsum of the tongue for stimulating the dorsum of the tongue to compress against the palate, will eventually result in closure of the pharyngeal airway, therefor actually inhibiting mouth and/or nasal breathing. As such, the device of Tepper causes a constriction of the airway as the dorsum contacts the palate.

Further Tepper teaches a device for statically positioning the tongue in a correct position, however, does not provide repetitive resistance exercise, as may be desired for strength training and cessation of use of a mouth-engaged appliance during sleep.

US Pub. No. 2007/0037665A1 to Robbins et al. teaches an oral resistance exercise device to improve the disorders of speech and swallowing. The device includes an upper lever portion and lower lever portion which are pivotally engaged at a central portion and include resistance means at one end and mouth engaging means at the other end. The user repetitively mechanically compresses the two levers together between their tongue and hard palate against the resistance for exercising the tongue and other muscles. However, the device includes a bulky portion which is inserted into the user's mouth and lacks proper oral ergonomics for comfortable periodic employment. The device additionally includes a portion which extends outside the user's mouth. Further, the device does not include means for maintaining proper proprioceptive sense and may easily be improperly employed.

US Pub. No. 2006/0124137A1 to Fong teaches an isometric exercise method and program for treating sleep apnea and snoring. The method includes isometric muscle manipulation of the tongue and jaw for strengthening the muscles related to airflow. However, without the provision of a resistance means, strengthening is highly limited.

As such, there is a continuing unmet need for an oral appliance device which provides a means for repetitive strength exercising of the pharyngeal muscles of a patient. Through repetitive use such a device should result in a natural forward positioning of the tongue within the throat by toning the muscles involved in tongue motion. Such a device, by strengthening and toning the muscles of the tongue, should lessen the overall size thereof due to toning. Through toning and strengthening such a device should reduce the narrowing of the pharyngeal airway during sleep. Such a device should advantageously engage fully into the mouth and provide means for comfortable removable engagement as needed for periodic exercise. Still further, such a device should provide variable means for exercising the pharyngeal dilators as toning occurs, while maintaining the pharyngeal airway substantially open.

The forgoing examples of related art and limitation related therewith are intended to be illustrative and not exclusive, and they do not imply any limitations on the invention described and claimed herein. Various limitations of the related art will become apparent to those skilled in the art upon a reading and understanding of the specification below and the accompanying drawings.

### SUMMARY OF THE INVENTION

The device herein disclosed and described provides a solution to the shortcomings in prior art and achieves the above noted goals through the provision of an oral appliance device for exercising and strengthening of the pharyngeal muscles. The device is configured to provide variable resistance exercise resulting in a strengthening of the pharyngeal muscles, especially the pharyngeal dilators, through repetitive resistance exercise. The device generally includes an oral appliance which engages on the palatal portion of the user's mouth. The device is maintained in an as used position preferably via retainer clasps extending into the lower arch or maxillary teeth or other means for removable engagement positioned upon the palatal portion of the mouth. It is possible the device may be engaged with the upper arch also.

It is noted that the terms, ‘posterior’, ‘anterior’, ‘forward’ and ‘rearward’, ‘front’, ‘rear’, ‘left’, and ‘right’ refer herein to the anatomical directions when considering the device in the as used position engaged to the user's palate. The use of these terms with various components should therefor be easily understood by a person skilled in the art as related to orientation, direction, and disposition.

In accordance with a first preferred mode, the body of the device has first and second (right and left) lateral sides which are formed to engage the lingual surfaces of the maxillary teeth of the lower arch and conform to the curvature and contour thereof. Means for positioning the device to an as used position for contact by the tongue for exercise is provided by a contoured engagement with either the lower or upper dental arch.

In a preferred mode, the lateral sides of the body of the device providing engagement with one dental arch are configured to engage the premolars and the molars and terminate at respective distal ends which are substantially adjacent to the incisors. However, it is noted that other configurations providing secured engagement to the palate, which one skilled in the art will recognize as suitable for the intended purpose, may be employed and are anticipated.

There is preferably also included a palatal bridge member which spans across the palate of the user when in the as used position. The bridge member is preferably engaged at the posterior ends of the left and right sides and communicates therebetween to provide rigidity and structural support for the device.

Additionally, there may be included one or a plurality of retainer clasps extending from the lateral sides of the body which are adapted for an engagement with adjacent teeth (i.e. molars, premolars, or both) for secured engagement of the device in the as used position.

The body of the device is preferably customized for an individual user by a qualified dental professional by molding impressions of the teeth and recording in a conventional manner or using a 3D x-ray to provide dimensions for 3D printing or other means for formation using 3D renderings. A dental laboratory then fabricates the device based on the impressions or the digital data provided by the dental professional using a suitable material which provides a comfortable feel to the patient and which may be readily cleaned or sterilized between usages.

In yet another particularly preferred method, pre-fabricated impression trays can be provided in a variety of sizes and shapes. The user or a qualified dental professional selects the one which best fits the user's mouth anatomy. This is also a conventional impression procedure where the dental professional prepares a customized version of the appliance using this tray to mold the impressions of the teeth and actual tongue shape of the patient.

In this mold mode a thermally responsive material is placed in the tray and the entire tray with liner is submerged in hot water so the liner softens. When placed in the mouth of the patient, and the patient bites down, the liner then directly forms to and around the patient's teeth. This process is commonly referred to as the ‘boil-and-bite’ process. When cooled the liner material is set retaining an impression of the patient's teeth and mouth and allows for subsequent routine placement into the mouth.

In another method for forming the invention, the body of the device is fabricated directly in the mouth of the patient using a two-part putty that hardens in a short period of time upon mixing of the two parts. A quantity of the putty is prepared and the hardening process is initiated by mixing the two parts together. That mixture is then placed into the impression tray, again in a conventional procedure. The entire form with putty is placed into the mouth of the patient, the teeth and tongue positioned appropriately. Once the putty has become firm, the entire assembly is removed from the patient's mouth and excess material making up the form is removed by processes such as cutting, grinding, or sanding.

Should 3D renderings be employed for formation of the body and device, a 3D x-ray is performed of the patient's mouth. The 3D data is then employed by the dental professional, or a lab, to either use a 3D printer to make the device or a mold to form the device to patient mouth dimensions.

Means for exercising the pharyngeal dilators while maintaining the pharyngeal airway open is provided by a pivoting or rotationally engaged tongue engaging member. The tongue engaging member, herein also referred to simply as the tongue member, is a substantially planar member having lingual and palatal opposing surfaces, a distal (anterior) edge, proximal (posterior) edge, and lateral side edges. The lingual surface of the tongue member is configured for an engagement with the dorsum of the tongue while the palatal surface is configured for an engagement with the palate. The tongue member is preferably sized to extend from the dorsal surface of the tongue to the tip of the tongue.

In still an additionally preferred method, employing the boil-and-bite process, the tongue member of the device additionally includes this aforementioned thermally responsive material. In this manner the user may additionally form the tongue member to a specified geometry. Added utility and advantage over prior art is found in that the boil-and-bite process is conventionally employed only with the teeth-receiving portions of such dental orthotics and not so with tongue related portions. A user is essentially provided with unlimited ability to customize the fit and feel of the device within their mouth. This mode of the device may be providable to users as a one-size-fits all model, where customization is done at home.

Using means for rotational or a pivoting engagement to the proximal (posterior) edge of the tongue member pivots in relation to the bridge member to allow deflection of the tongue member. Examples of a pivoting engagement means can include one or a plurality of mechanical hinges, living hinges, pivots, or the like. Briefly, living hinges are comprised of a soft, durable, and flexible plastic or rubber and can advantageously be formed unitarily with tongue and bridge portions, and are thus preferred in most instances for ease of manufacture. Further, the living hinge can advantageously be formed with a smooth surface for added comfort. However, in some individuals a mechanical hinge may work better due to their strength and the number of repetitions the device will make over time wearing out the living hinge.

By employing rotational or pivoting engagement disposed at the posterior end of the tongue member, and provided that the tip and anterior portion of the tongue is in an engagement with the tongue member, during operative use on each repetition of the tongue to pivot the tongue member, the user's tongue is urged in the proper forward thrust position with the tip of the tongue on the incisal edges. As such, the forward thrust of the tongue during employment of the device maintains the user's airway open, and is of advantage over prior art. For example, the above noted device to Tepper (U.S. Pat. No. 5,052,409) is configured to engaged only the posterior portion of the tongue dorsum and not the anterior portion/tip, and as a result will urge the tongue slightly or moderately toward the back of the mouth which results in closure of the pharyngeal airway at all times.

The tongue member may be customized for an individual user by molding impressions of the tongue and palate. A dental laboratory then fabricates the tongue member based on the data provided by the dental professional using a suitable material which provides a comfortable feel to the patient and which may be readily cleaned or sterilized between usages.

Further, as an advantage over prior art, projections of the rugae of the palate can be formed on the lingual surface of the tongue member to provide a means for proprioceptive feel and therefor proper tongue positioning during use.

In accordance with one preferred mode, resistance means is provided by forming the rotational engagement means to bias the tongue member away from the palate and toward the tongue. This can be accomplished by employing a spring loaded hinge or forming a living hinge to impart a bias in the desired direction.

Also in the preferred mode of the device, a means for resistance to pivoting of the tongue member may be provided. Depending on whether a living hinge or mechanical hinge is used, this means for resistance may vary. However, currently an elastic member or band, which extends over the palatal surface of the tongue member, provides such resistance and is well tolerated in the mouth environment. The ends of the elastic band are preferably engaged to hooks or mounting means disposed on the respective opposing sides of the body of the device. As such, the elastic band is configured to bias the tongue members against the tongue when in the as used mode. This elastic member means for resistance may also be variable and allow the user to employ larger or more resistive elastic members as they progress in their exercise regimen. The device may be provided as a kit, where a plurality of elastic members is provided with the body and tongue member such that the user may choose one of the plurality where each has a differing resistance level.

Should a hinged mode of the device be employed, or in some case a living hinge, a means for resistance to pivot of the tongue member may be provided by a spring engaged at the hinge point with the body. Alternatively, springs may be engaged to both opposing sides of the body and to the distal end of the tongue member to provide resistance to pivoting during use. These springs may vary in resistance and also be provided as a kit with a plurality of differentiating springs of variable resistance to allow the user to choose.

For all preferred modes, the device allows a user to perform a repetitive resistence exercise by repetitively manipulating their tongue to compress or pivot the tongue member against the resistive force provided by means for resistance to the pivoting of the tongue member toward the palate. This exercise is advantageously performed while the tongue is in a forward thrust position, and the airway is maintained substantially open through the entire exercise. As a further advantage over prior art, and adding utility, the device may include one or a plurality of targeting means or targets which provide proprioceptive responses to the user that their tongue is positioned on the contact point with the tongue member so the device is employed as intended in the as used position to allow the user with the tongue in the proper contact point adjacent the distal end, to employ the device for exercise.

In accordance with at least one preferred mode, the distal (anterior) edge of the tongue member includes a cutout or notched portion which communicates with the incisive papilla (a projection on the palate near the incisors) providing a first targeting means. Therefor, in the as used position the user can position the tip of their tongue at or near the notch portion providing proprioceptive feel of the incisive papilla and an indication of a correct initial thrust positioning of the tongue. Further, with the tongue in a forward position, the airway is maintained open and the user is permitted to breathe normally.

Next, a second targeting means is provided by a raised projection or bump engaged at or near the proximal edge on the lingual surface of the tongue member and projecting toward the tongue. As the user presses their tongue against the tongue member, and the tongue member compresses against the resistance means, the projection provides a proprioceptive response that the dorsum of the tongue is in the proper position for the finish of the exercise performance. Additionally, the airway is maintained substantially open.

Further, a third targeting means is provided by a raised projection or bump engaged to the bridge portion of the device. Therefor, upon completing the compression of the tongue member against resistance means where the tongue member contacts the palate, if the user continues to compress the tongue member, a contact of the user's tongue with the projection provides a proprioceptive response that the posterior dorsal surface of the tongue proximate the location of the bridge portion has moved beyond the proper exercise envelope. At this point in the exercise, it may be noted by the user that the airway is constricted or closed due to positioning of the tongue engaged the raised projection. This is considered past the exercise envelope and as such will indicate to the user that the motion was completed incorrectly.

It is noted that the means for targeting of the tongue to a contact point on the tongue member may additionally include means for gustatory response. This can include engaging a gustatorial stimulant at or near the targeting means. Thus, the target component may be a surface point providing a tactile response to the tongue or gustatory response so the user has a proprioceptive sensation they have their tongue on the contact point.

It should also be noted that the device has applications for other user reasons for a strengthening of the muscles of the mouth and for tongue dexterity. Such is particularly desirable by some individuals during sexual intercourse and as such the device herein is anticipated for employment for any use where a strengthening of tongue muscles and for increasing tongue dexterity is desirable.

With respect to the above description, before explaining at least one preferred embodiment of the herein disclosed invention in detail, it is to be understood that the invention is not limited in its application to the details of construction and to the arrangement of the components in the following description or illustrated in the drawings. The invention herein described is capable of other embodiments and of being practiced and carried out in various ways which will be obvious to those skilled in the art. Also, it is to be understood that the phraseology and terminology employed herein are for the purpose of description and should not be regarded as limiting.

As such, those skilled in the art will appreciate that the conception upon which this disclosure is based may readily be utilized as a basis for designing of other structures, methods and systems for carrying out the several purposes of the present disclosed device. It is important, therefore, that the claims be regarded as including such equivalent construction and methodology insofar as they do not depart from the spirit and scope of the present invention.

As used in the claims to describe the various inventive aspects and embodiments, “comprising” means including, but not limited to, whatever follows the word “comprising”. Thus, use of the term “comprising” indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of’ is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

Objects of the invention will be brought out in the following part of the specification, wherein detailed description is for the purpose of fully disclosing the invention without placing limitations thereon.

### BRIEF DESCRIPTION OF DRAWING FIGURES

The accompanying drawings, which are incorporated herein and form a part of the specification, illustrate some, but not the only or exclusive, examples of embodiments and/or features. It is intended that the embodiments and figures disclosed herein are to be considered illustrative rather than limiting. In the drawings:

FIG. 1 shows a perspective view of a particularly preferred mode of the device providing an oral appliance having a rotationally engaged tongue member for use in repetitive resistance exercise.

FIG. 2 shows a top view of the device.

FIG. 3 shows a view of the device in the as used mode engaged to the palate of a user's mouth.

FIG. 4 depicts a mode of the device herein employing elastic bands as a means to impart resistance during exercise.

FIG. 5 depicts the device of FIG. 4 showing opposing mounts for maintaining the elastic bands in position.

FIG. 5a depicts a mode of the device wherein a flexible resistant member provides both a hinged engagement and means to resist movement by the tongue.

FIG. 6 depicts a mode of the device showing the employment of hinges for a pivotal engagement and springs in either of two positions providing means for resistance to pivoting.

FIG. 7 depicts one mode of the method for providing exercise to sleep apnea patients and others needing resistance exercise of the throat muscles.

### DETAILED DESCRIPTION OF THE PREFERRED

### Embodiments of the Invention

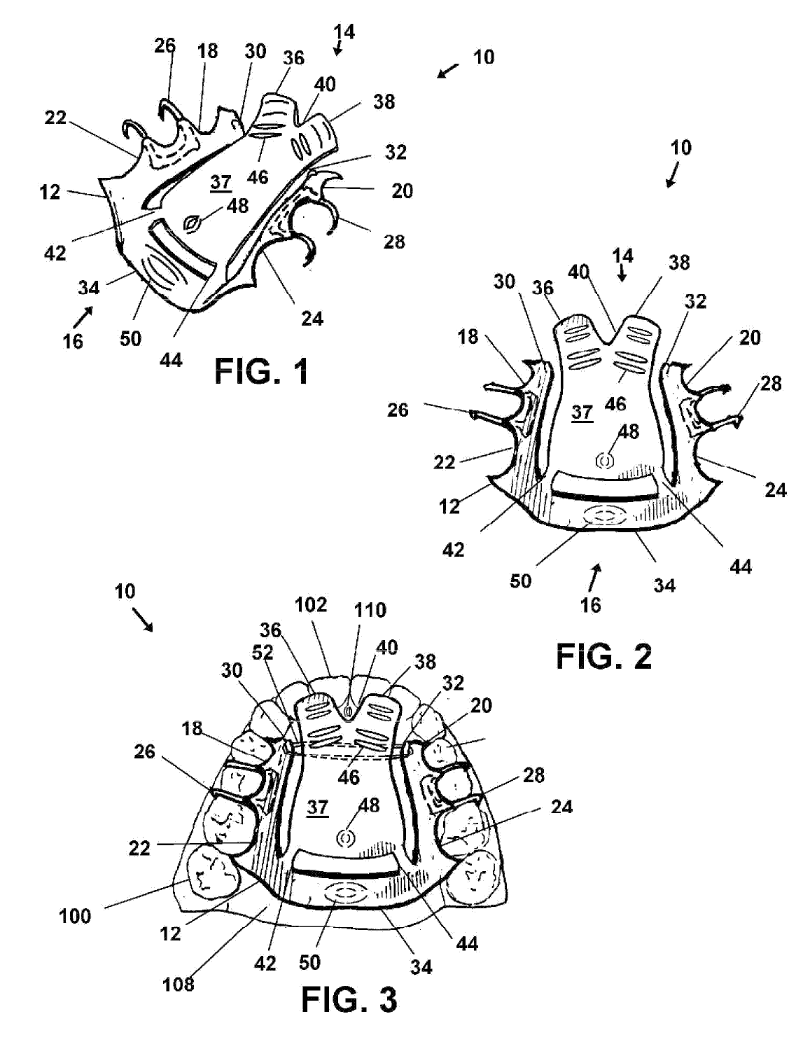

Now referring to drawings in FIGS. 1-7, which depict some preferred modes of the device 10 and method herein, and wherein similar components are identified by like reference numerals, there is seen in FIG. 1, FIG. 2, and FIG. 3 views of a particularly preferred mode of the device 10. The device 10 provides an oral appliance body 12 for engagement onto the lower arch and palate 108 of A user's mouth, having a distal (anterior) end 14 and a proximal (posterior) end 16.

The body 12 provides means for a pivoting positioning of the tongue member 36 in the mouth in a position where it is contractible by the user's tongue for pivoting during exercise. The means the body 12 may engage with the upper or lower dental arch as long as it positions the tongue member 36 in proper positioning for pivoting during exercise. Currently the lower dental arch is preferred as the frictional engagement of the body 12 is easier on the user's mouth when so engaged.

The body 12 employs means for operative engagement between both opposing rows of user teeth of a dental arch, using first and second lateral side portions 18, 20 respectively each configured with contours 22, 24 adapted to engaged the lingual surfaces of the maxillary teeth 100. The contours 22, 24 may be customized for an individual user by molding impressions of the teeth 100 and recording in a conventional manner. The body 12 is thus configured to provide a means for pivoting engagement to the tongue member 36 with the tongue member 36 substantially centered in the mouth so the distal end is contactable by the tongue of the user. Frictional or mechanical engagement between the teeth of the lower arch of the user is currently preferred.

The various components of the device 10 disclosed herein can be formed of conventional materials such as synthetic polymers methacrylate or acrylic, however can be formed of any material suitable for the purposes set forth in this disclosure.

Engagement to the user's palate is provided by one or a plurality of retainer clasps 26, 28 which are engaged to the respective sides 18, 20 of the body 12 and extend therefrom. The clasps 26, 28 can be conventional retainer clasps known in the art. In other modes, however, means for engagement to the palate 108 may be provided by forming the sides 18, 20 of the body 12 of the device 10 to impart an outward bias against the lingual surfaces of the maxillary teeth 100 and provide a biased engagement. Still, it is noted and anticipated that one skilled in the art may recognize other means for engagement to the palate, which are within the scope of the invention, and are considered part of this patent.

At the proximal end 16 of the engagement portion of the device 10, there can be seen a bridge member 34 communicating between and engaging the proximal ends 16 of the side portions 18, 20. The bridge member 34 spans the palate 108 and is preferably curved and contoured to the shape of the user's palate. The bridge 34 provides structural support and rigidity for the body 12 and serves other purposes set forth shortly.

Further, there are included hook portions 30, 32 located at the terminating distal ends of the sides 18, 20 of the body 12 of the device 10. Briefly, these hook portions 30, 32 provide mounting means for the engagement of one or a plurality of elastic bands 52 (FIG. 5) which provide an adjustable means for resistance to deflective movement of the pivoting or movably engaged tongue member 36 by the force from the user's tongue thereon. However, other means for mounting of the elastic bands 52 may be employed and are anticipated.

The tongue member 36 is substantially planar and may be slightly curved or contoured to provide ergonomic engagement with the lower side of the user's tongue during use. The distal edge 38 of the tongue member 36 may include a recessed or notched portion 40 which in use provides a tactile first target for a proprioceptive feel of the incisive papilla 110 (a projection on the palate 108 near the incisors 102) so the tongue is properly on the contact point. Detailed explanation of the preferred means for targeting and use thereof are described shortly.

The proximal end of the tongue member 36 is rotationally, or moveably, engaged to the U-shaped body 12 to a position with one edge immediately adjacent bridge member 34. Means for pivoting engagement of the tongue member 36 to the body 12 can include one or a plurality engagement components from a group of pivoting components including hinges, pivots, a flexible member engaged therebetween, or a living hinge 42 and 44 such as in FIG. 2. A living hinge 42 and 44 would be formed from a section of the body 12 and tongue member 36 extending between the body 12 and the tongue member 36, which would be flexible and allow movement of the tongue member relative to the body 12.

As shown in FIGS. 1-3, one or a plurality of living hinges 42, 44 can be formed. The living hinges 42, 44 can be formed of a soft, durable, and flexible plastic or rubber and can advantageously be formed unitarily with tongue 36 and bridge 34 members, such as through a two shot injection molding process. However, in other modes of the device employing conventional hinges or pivots, the hinges or pivots may be engaged to the device 10 by conventional techniques known in the art.

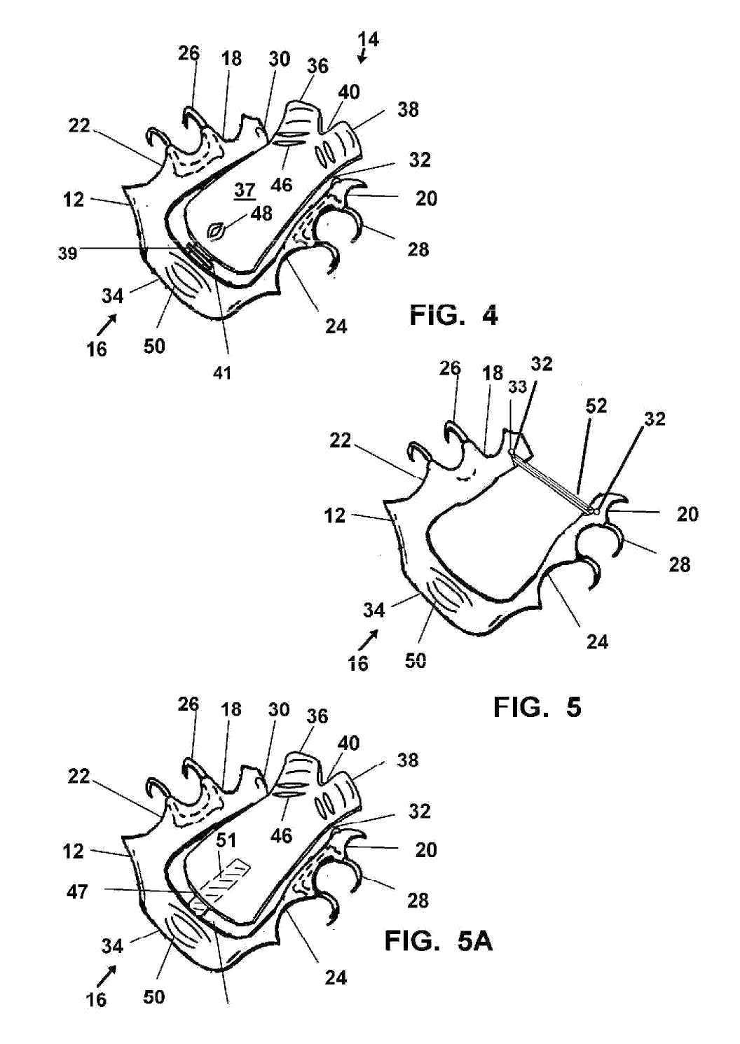

In another preferred mode, the means for pivoting resistance can be provided by forming the living hinges 42, 44, of material or a thickness or both, to impart an upward bias against the tongue member 36, away from the user's palate, and toward their overhead tongue. Alternatively, in another mode, the resistance to pivoting can be provided by employing a hinge 41 (FIG. 4) which is a spring 39 loaded hinge 41 to resist movement of the tongue portion 36. Other means for resistance to pivoting of the tongue member 36 which are suitable for the intended purpose may also be employed within the scope of the invention, and are anticipated, with variable means for resistance being particularly favored so as to allow the user to adjust to need.

Added utility is provided through the employment of means for tactile targeting for tongue contact which provides proprioceptive responses indicating that the device 10 is being employed properly. In accordance with at least one preferred mode, the distal (anterior) edge 38 of the tongue member 36 includes a cutout or notched portion 40 which communicates with the incisive papilla 110 providing a first targeting means.

Therefor, in the as used position, using a tactile sense or sense of feeling on their tongue, the user can ascertain with the tip of their tongue and position the tip of their tongue at or near the notch portion 40. This positions the tongue in proper position for a forward thrust and provides proprioceptive feel of the incisive papilla 110 additionally indicating a correct initial positioning of the tongue. Further, by maintaining this forward tongue thrust during employment, the airway is maintained open and pharyngeal dilators are especially exercised.

Next, a second means for targeting tongue position properly is provided by a raised projection or bump 48 engaged at or near the proximal edge on the lingual surface 37 of the tongue member 36 and projecting therefrom. In use during proper tongue positioning, as the user presses the bottom surface of their tongue against the contact surface of the tongue member 36, and further pressing to overcome the means for resistance to movement, the projection 48 provides a proprioceptive response that the dorsum of the tongue, allowing the user a tactile means, without looking, to ascertain that their tongue is in the proper alignment on the contact surface of the tongue member 36.

A third means for targeting a proper positioning of the user's tongue, may be provided by positioning a raised projection or bump 50, engaged upon the bridge member 34 of the U-shaped body of the device 10. If the user inadvertently continues to press their tongue against the tongue member 36 causing over rotation, or after it can no longer move, the projection 48 provides a proprioceptive response that the tongue member 36 has been compressed too far by the user's tongue. Further, the user will also note, in addition to proprioceptive response, that their airway is substantially constricted with the tongue in this position, further providing an indication that the tongue member 36 has been compressed too far and that they should back off slightly. Continuous contact with this bump 50 also provides a means for signaling the user that they should increase the resistance provided by the means for resisting pivoting movement of the tongue member, such as the elastic bands 52. A kit of elastic bands 52 can be provided with the device 10, which different members of the kit have respective different resistance to stretching, and thus provide differing resistance to pivoting of the tongue member 36 by the user. Changing the elastic bands 52 to less elastic bands from the kit, thus provides more resistance during use.

Further, projections shown as rugae 46 of the palate can be formed on the lingual surface 37 of the tongue member 36 to provide additional means for proprioceptive feel and therefor proper tongue positioning during use.

FIG. 4 shows a mode of the device 10 wherein the tongue member 36 is engaged to the body 12 using a single hinge 41. As noted above, the hinge 41 may also be a spring 39 loaded resistance type hinge 41 which provides resistance to rotation in one direction thereby providing means for resistance to pivoting. The spring loaded hinge 41 can thus provide means for resistance to pivoting of the tongue portion 36, downward or toward the user's chin, by itself, or in combination with the elastic bands (FIG. 5). In this mode of the device, the tongue portion 37 may be permanently or removably engaged to the hinge 41 by insertion of one member of the hinge 41 in a formed slot 47 in the tongue member 36. The slotted engagement would be frictional and allow for removal and replacement of the tongue member 36 as needed without replacing the body 12. As with other modes of the device 10, with springs 39, the resistance provided may vary and a kit of a plurality of engageable springs of differing resistance may be provided.

FIG. 5 depicts a mode of the device 10 herein showing the tongue portion 36 removed and a positioning of elastic members or elastic bands 52 which provide means for resistance to pivoting of the tongue member, engaged upon opposing hook portions 30 and 32 which are engaged at one end to the body 12. The hook portions each have a rounded distal end 33 which has a diameter exceeding that of the member forming hook portions 30 and 32. This rounded portion 33 is particularly preferred as a means to prevent nicking or puncture of the patient's tongue, and as a means to maintain the elastic bands 52 or elastic members in position encircling the hook portions 30 and 32, but prevented from sliding from the respective distal ends thereof.

FIG. 5a depicts a mode of the device wherein a flexible resistant elastic member 51 provides both a means for resistance to pivoting of the tongue portion 36 to the body 12 and provides a means to resist movement downward toward the chin, (or rotational around the axis running through the body 12 at the attachment point.) Engagement of the elastic member 51 may be by frictional engagement in a slot 47 to allow for removal and replacement of the tongue portion 36, or may be permanent.

It should be noted, engagement in a slot 47 with the tongue portion 36 and with the body 12, is preferable as the elastic member 51 may be provided in a kit of engageable elastic members 51 with each having a differing resistance to deflection when engaged between the tongue portion 36 and the body 12. This allows the user to change the member 51 when more resistance to pivoting or downward movement is desired.

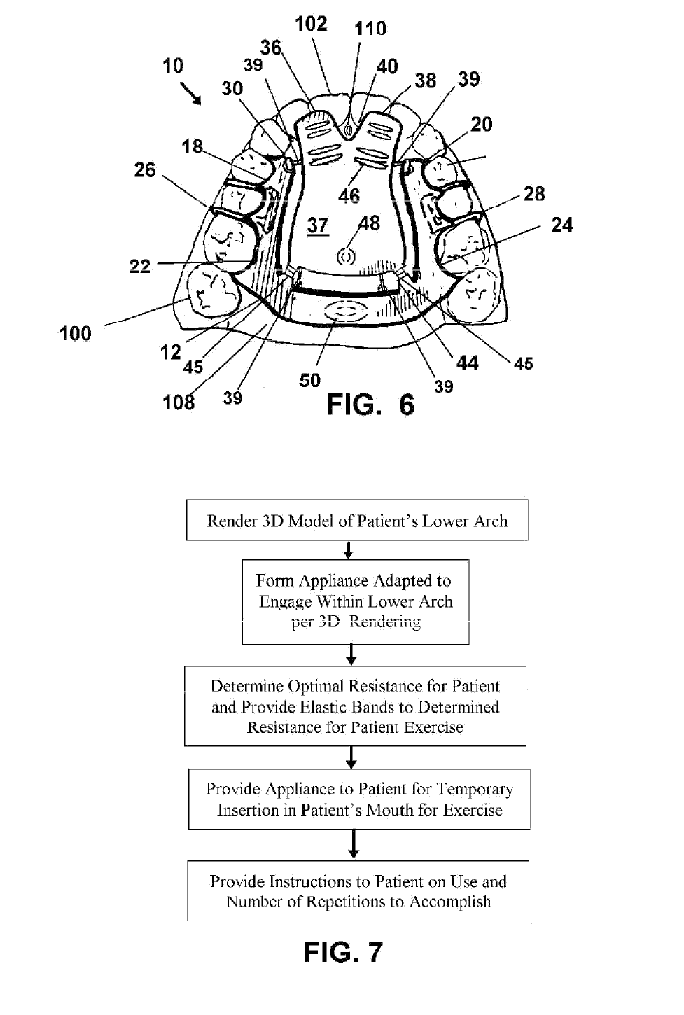

FIG. 6 depicts a mode of the device 10 showing the employment of hinges 45 for a pivotal engagement of the tongue member 36 and springs 39 which may be employed in either or both depicted positions as means for imparting resistance to pivoting of the tongue member 36. As noted above, the springs 39 may be used if elastic bands are not desirable and the springs 39 may be provided in a kit having a plurality of springs 39 in varying resistance to allow the user or professional to implement a change when more or less resistance to pivoting of the tongue member 36 by the force of the user's tongue is desired. The hinges 45 provide a means for pivoting engagement of the tongue member 36 might also be employed instead of the living hinge if such is desirable or required.

In FIG. 7 is shown a representation of the method of the system herein. In a first step, once a patient is determined to have sleep apnea, or another medical condition which will be treated by the device 10, a 3D rendering of the area between the lower arch and top of the palate of the patient is taken. This may be by 3D x-ray or using dental molding material placed in the area being rendered. A 3D x-ray may be preferable since the digital file may be electronically communicated to a lab or a forming machine which will use the topographical rendering to form the body 12 and tongue portion 36 in dimensions complimentary to the curves and area of the positioning between both sides of the lower arch.

In the second step, the body 12 and tongue portion 36, sized to the patient are formed, using molding, 3D printing, machining, or other means for using the 3D rendering to form the body 12 and tongue portion 36.

In a subsequent step, a dental professional will determine the optimal resistance to be provided by the device 10 and provide the appropriate elastic bands, flexible member, spring loaded hinge, singularly or in combination, to initiate use by the patient with the proper initial resistance.

With the initial resistance determined, the device is provided to the patient with the proper resistance components, for a fitting and if the fit is acceptable, in a final step, instructions for use are provided including any instructions on when to change or increase resistance of the deflection of the tongue member 36. As noted the means for resistance may be provided to the user in kit form with interchangeable components in the kit to allow replacement with components of less or more resistance to deflection. The resistive components as noted above may be either or both from a group of resistive components including elastic bands 52, elastic members 51 and springs 39 which when operatively engaged between the body 12 and the tongue member 36, provide a resistive biasing or resistive force to pivoting of the tongue member 36 by contact with the user's tongue with the device 10 in the as used position where the tongue member 36 is pivotally positioned between the lower arch of a user's mouth.

The device 10 and method herein, while focusing on treatment for sleep apnea through throat muscle exercise and toning is also employable for patients needing re-acquisition of their natural or habitual bite and to correct mis-position side effects of wearing conventional oral appliances for sleep apnea.

Further, upon reading this disclosure, those skilled in the art will surely recognize various other means to provide an oral appliance for application of resistance exercise using a deflecting tongue resisting member, which are considerably or slightly different those disclosed. As such, any device configured for positioning in the patient's mouth and providing resistance to deflection of a tongue-contacted member, are considered within the scope and intent of the invention herein, and are anticipated withing the scope of this patent.

It is additionally noted and anticipated that although the device is shown in its most simple form, various components and aspects of the device may be differently shaped or slightly modified when forming the invention herein. As such those skilled in the art will appreciate the descriptions and depictions set forth in this disclosure or merely meant to portray examples of preferred modes within the overall scope and intent of the invention, and are not to be considered limiting in any manner.

While all of the fundamental characteristics and features of the invention have been shown and described herein, with reference to particular embodiments thereof, a latitude of modification, various changes and substitutions are intended in the foregoing disclosure and it will be apparent that in some instances, some features of the invention may be employed without a corresponding use of other features without departing from the scope of the invention as set forth. It should also be understood that various substitutions, modifications, and variations may be made by those skilled in the art without departing from the spirit or scope of the invention. Consequently, all such modifications and variations and substitutions are included within the scope of the invention as defined by the following claims.

What is claimed:

1. An oral exercise apparatus comprising:
an elongated member having a proximal end and distal end;
means for pivoting engagement of said proximal end of said elongated member, between the teeth of a lower dental arch of the mouth of a user in an as used position;
a contact point adjacent said distal end of said elongated member, said contact point positioned for a contact with the distal end of a user's tongue with said elongated member in said as used position; and
said contact with said user's tongue imparting a pivoting of said elongated member toward the palate of said user within said lower dental arch, whereby said user may exercise their pharyngeal muscles by said pivoting of said elongated member.

2. The oral exercise apparatus of claim 1 wherein said means for pivoting engagement of said proximal end of said elongated member comprises:
a body, said body having a proximal end and distal end and having side edges extending therebetween; and
means for removable engagement of said side edges with adjacent said teeth of said lower dental arch when in said as used position;
a pivoting engagement of said proximal end of said body with said proximal end of said elongated member.

3. The oral exercise apparatus of claim 3 wherein said pivoting engagement of said proximal end of said body with said proximal end of said elongated member, comprises:
any one of a group of pivoting engagements from a group including a living hinge formed between said body and said proximal end of said elongated member, and a hinge engaged between said body and said proximal end of said elongated member and a pivoting component engaged between said body and said proximal end of said elongated member.

4. The oral exercise apparatus of claim 1 additionally comprising:
means for resistance to said pivoting of said tongue member.

5. The oral exercise apparatus of claim 2 additionally comprising:
means for resistance to said pivoting of said tongue member.

6. The oral exercise apparatus of claim 3 additionally comprising:
means for resistance to said pivoting of said tongue member.

7. The oral exercise apparatus of claim 4 wherein said means for resistance to said pivoting of said tongue member is one or a combination of a group of resistive components including elastic bands, elastic members, and springs.

8. The oral exercise apparatus of claim 5 wherein said means for resistance to said pivoting of said tongue member is one or a combination of a group of resistive components including elastic bands, elastic members, and springs.

9. The oral exercise apparatus of claim 6 wherein said means for resistance to said pivoting of said tongue member is one or a combination of a group of resistive components including elastic bands, elastic members, and springs.

10. The oral exercise apparatus of claim 7 additionally comprising:
a target positioned adjacent said distal end of said elongated member, said target defining said contact point;
said target configured to provide one of a tactile or gustatory response when in a contact with said user's tongue, whereby said user may ascertain a contact of said tongue with said contact point.

11. The oral exercise apparatus of claim 8 additionally comprising:
a target positioned adjacent said distal end of said elongated member, said target defining said contact point;
said target configured to provide one of a tactile or gustatory response when in a contact with said user's tongue, whereby said user may ascertain a contact of said tongue with said contact point.

12. The oral exercise apparatus of claim 9 additionally comprising:
a target positioned adjacent said distal end of said elongated member, said target defining said contact point;
said target configured to provide one of a tactile or gustatory response when in a contact with said user's tongue, whereby said user may ascertain a contact of said tongue with said contact point.

13. The oral exercise apparatus of claim 7 additionally comprising:
a plurality of each resistive component in said group of resistive components, each of said resistive components in said plurality providing a different resistive force, whereby said resistive force may be changed by changing a said resistive component to one of more or less resistance.

14. The oral exercise apparatus of claim 8 additionally comprising:
a plurality of each resistive component in said group of resistive components, each of said resistive components in said plurality providing a different resistive force, whereby said resistive force may be changed by changing a said resistive component to one of more or less resistance.

15. The oral exercise apparatus of claim 9 additionally comprising:
a plurality of each resistive component in said group of resistive components, each of said resistive components in said plurality providing a different resistive force, whereby said resistive force may be changed by changing a said resistive component to one of more or less resistance.

16. The oral exercise apparatus of claim 10 additionally comprising:
a plurality of each resistive component in said group of resistive components, each of said resistive components in said plurality providing a different resistive force, whereby said resistive force may be changed by changing a said resistive component to one of more or less resistance.

17. The oral exercise apparatus of claim 11 additionally comprising:
a plurality of each resistive component in said group of resistive components, each of said resistive components in said plurality providing a different resistive force, whereby said resistive force may be changed by changing a said resistive component to one of more or less resistance.

18. The oral exercise apparatus of claim 12 additionally comprising:
a plurality of each resistive component in said group of resistive components, each of said resistive components in said plurality providing a different resistive force, whereby said resistive force may be changed by changing a said resistive component to one of more or less resistance.
